(19) United States
(12) Patent Application Publication
(10) Pub. No.: US 2021/0400931 A1
(43) Pub. Date: Dec. 30, 2021

(54) METHOD FOR CONSTRUCTING LUNG CANCER ANIMAL MODEL

(71) Applicant: SUN YAT-SEN UNIVERSITY, Guangdong (CN)

(72) Inventors: Mengfeng LI, Guangdong (CN); Yan SUN, Guangdong (CN); Jueheng WU, Guangdong (CN); Junchao CAI, Guangdong (CN)

(73) Assignee: SUN YAT-SEN UNIVERSITY, Guangdong (CN)

(21) Appl. No.: 16/963,511

(22) PCT Filed: Feb. 25, 2020
(86) PCT No.: PCT/CN2020/076640

§ 371 Date: Jul. 21, 2020

(30) Foreign Application Priority Data
Feb. 17, 2020 (CN) 202010097296.9

(51) Int. Cl. A01K 67/027

(52) U.S. Cl. CPC A01K 67/027; A01K 2267/0331

(57) ABSTRACT

A method for constructing a lung cancer animal model. According to the method, a pathogenic substance of a lung cancer is atomized into atomized particles, and an animal inhales the atomized particles in an inhaling mode, so as to construct a required lung cancer animal model.

### TECHNICAL FIELD

The present invention belongs to the field of biological medicine technologies, and more particularly, relates to a method for constructing a lung cancer animal model.

### BACKGROUND

As the most common malignant tumor with a highest mortality in the world, a lung cancer seriously threatens human life and health. Therefore, it is especially important to develop a new specific and effective target drug and an innovative treatment method. Researches on pathogenesis of the lung cancer and development of related therapeutic drugs are top priorities, and cannot be separated from a lung cancer animal model.

Tyler Jacks from Massachusetts Institute of Technology first proposed a method for constructing a lung cancer model reported in the existing research which includes intranasal instillation and intratracheal instillation inhalation, and is a classic method for constructing a lung cancer model (Conditional mouse lung cancer models using adenoviral or lentiviral delivery of Cre recombinase. Nature Protocol, 2009, Vol 4, No. 8, 1064-1072. Authors: Michel DuPage & Alison L Dooley & Tyler Jacks. Massachusetts Institute of Technology) at current.

However, this method has a lot of defects and limitations as follows: (1) the method is time consuming to operate and needs special training, and an operator needs to have high professional skills; (2) the method has a risk of a digestive tract tumor caused by instilled liquid entering a stomach through an oral cavity, thus leading to failed model construction; (3) death of a mouse by hemorrhage or suffocation is caused by improper instillation or inhalation, thus leading to failed model construction; (4) there is no uniform standard for the lung cancer animal model because an accuracy of a drug or virus administration dosage by the intranasal instillation and the intratracheal instillation inhalation cannot be controlled; (5) sites where the instilled liquid enters lungs are nonuniform, so that a randomness of a human lung cancer cannot be more accurately simulated; (6) affected histological sites are not specific due to a limitation of an administration route according to the above method, so that an origin and a generating process of a lung cancer cell cannot be accurately simulated; and (7) standardization of the lung cancer model cannot be realized due to the above limitations.

### SUMMARY

The technical problem to be solved by the present invention is to overcome the above defects and deficiencies of the existing lung cancer model construction technology, and provide a method for constructing a standardized lung cancer animal model capable of more accurately simulating an incidence of human and constructing an origin and a generating process of a lung cancer cell as required, and the method is simple, stable and standard to operate.

An objective of the present invention is to provide a method for constructing a lung cancer animal model.

The above objective of the present invention is achieved by the following technical solutions.

According to a method for constructing a lung cancer animal model, an animal is subjected to a nebulization after a pathogenic substance of a lung cancer is atomized into atomized particles, so that the animal inhales the atomized particles in an inhaling mode.

Preferably, a size of the atomized particle is that: a mean median diameter (MMAD) is 2.9 μm; and a percentage of a particulate smaller than 5 μm is 76%.

Preferably, the nebulization is to continuously inhale for 15 minutes to 20 minutes.

Preferably, an atomization amount of the pathogenic substance of the lung cancer ranges from 2 ml to 8 ml.

Preferably, after inhaling the atomized particles, the animal is placed in an SPF environment to obtain a required lung cancer animal model.

Specifically and preferably, according to the method for constructing the lung cancer animal model according to the present invention, the animal is subjected to the nebulization after the pathogenic substance of the lung cancer is atomized into the atomized particles by using an atomization inhalation instrument, and operating parameters of the instrument are as follows:

pressure: 0.5 bar/50 kpa to 2.0 bar/200 kpa

atomization amount: 2 ml to 8 ml

working flow: 3.0 L/min to 6.0 L/min

atmospheric pressure: 500 hpa to 1060 hpa

atomization rate: 370 mg/min

size of particle: mean median diameter (MMAD): 2.9 μm; and percentage of particulate smaller than 5 μm: 76%.

According to the method for constructing the lung cancer animal model provided by the present invention, the standardized animal model simulating the origin and the generating process of the lung cancer cell can be constructed as required, since cells of non-small cell lung cancers originate from a terminal bronchiolar epithelium and an alveolar epithelium, the constructed lung cancer model simulates an origin of the cancer cell as much as possible. According to our method, a diameter of the atomized particle can be controlled to be about 2 μm to 3 μm (preferably 2.5 μm to 3 μm), and particles with the diameter can be finally positioned on the terminal bronchiole and the alveolar. Moreover, the atomization instrument can generate atomized particles with a uniform size at a constant speed, so that the atomized particles are uniformly distributed in all alveoli and terminal bronchioles. In aspects of an origin of a lung cancer tissue cell and a uniformity of a site of action, the method can be closest to simulate the generating process of a human lung cancer (an adenocarcinoma in non-small cell lung cancers). Moreover, the method is simple and easy to operate, and can simulate all processes such as starting, atypical hyperplasia, carcinoma in situ and invasive carcinoma of the lung cancer by controlling a virus concentration. The method has very important significance for studying key nodes in occurrence and development of the lung cancer and effective target drug control.

The constructed lung cancer model stimulates the origin of the cancer cell as much as possible, and we control the diameter of the atomized particle to be about 2.5 μm to 3 μm, then particles with the diameter can be positioned on the terminal bronchiole and the alveolar without affecting tissue cells of other sites. In the case that the diameter of the atomized particle ranges from 2 μm to 3 μm, the lung cancer stimulated by the constructed animal model is an adenocarcinoma in non-small cell lung cancers (derived from an alveolar epithelium cell and a terminal bronchial epithelial cell).

Due to particularity of anatomical structure of human lung tissue, particles inhaled from the outside can reach different sites due to different sizes, the particles with a size ranging from 5 μm to 10 μm can reach a main bronchus and a secondary bronchus, the particles with a size ranging from 3 μm to 5 μm reach the secondary bronchus and branched bronchi, and the particles with a size less than 3 μm reach a terminal bronchiole and an alveolus. Therefore, we can simulate different types of lung cancers and all stages in occurrence and development of each lung cancer by: firstly, controlling the diameter of the atomized particle; secondly, using different genetically engineered mice; and thirdly, controlling inhalation of different viruses and viruses with different concentrations, or a concentration of a chemical carcinogen.

In the case that a diameter of the atomized particle ranges from 5 μm to 10 μm, the obtained animal model simulates the lung cancer occurring on a main bronchus and a secondary bronchus, in the case that the diameter ranges from 3 μm to 5 μm, the obtained animal model simulates the lung cancer occurring on the secondary bronchus and branched bronchi, and in the case that the diameter is less than 3 μm, the obtained animal model simulates the lung cancer occurring on a terminal bronchiolar epithelium and an alveolar epithelium.

For example, in the case that the pathogenic substance of the lung cancer is an adenovirus carrying a Cre recombinase, and the diameter of the atomized particle ranges from 5 μm to 10 μm, the atomized particles are inhaled by Kras^{LSL-G12D};LKB1^{fl/fl} genetically engineered mice to construct lung cancer (a squamous carcinoma in non-small cell lung cancers) mouse models.

In addition, different stages of the lung cancer can also be stimulated by controlling a concentration of a virus drug. When the pathogenic substance of the lung cancer is an adenovirus carrying a Cre recombinase, a Kras oncogene of a lung epithelial cell can be activated; in the case that a virus concentration is 5×10⁵-5×10⁶, the obtained animal model simulates an early stage of the lung cancer; in the case that the virus concentration is 2.5×10⁷, the obtained animal model simulates a progressing stage of the lung cancer; and in the case that the virus concentration is 7.8×10⁹, the obtained animal model simulates an invasive carcinoma stage of the lung cancer. The foregoing well simulates various stages in the occurrence and development of the lung cancer.

The World Health Organization divides the lung cancers into a small cell lung cancer and non-small cell lung cancers according to an occurrence site of the lung cancer, a cell origin and a clinical characteristic, and the non-small cell lung cancers can be subdivided into an adenocarcinoma, a squamous carcinoma, a large cell lung cancer and a carcinoid cancer. Clinically, different therapeutic schemes are used for different types of lung cancers. Therefore, accurate simulation of different types of lung cancers in the present invention is particularly important for studying occurrence and development mechanisms of the lung cancer and targeted drug treatment.

The present invention has the following beneficial effects.

The present invention provides a method for constructing a more accurate and standardized lung cancer animal model, and the virus drug is atomized into particles with a specific parameter using the atomization instrument, so that the animal inhales the atomized particles in an inhaling mode to successfully construct the mouse lung cancer model. The method is simple and easy to operate without special training, and can be well popularized.

According to the method, aerosol liquid drops inhaled by an animal are controlled to range from 2 μm to 5 μm, and the aerosol liquid drops can be uniformly dispersed on the alveolar epithelial cells, so that the carried adenovirus thereof enters the epithelial cell and activates the Kras gene, thus site-specifically and accurately simulating and inducing occurrence of the lung cancer. An effective rate of constructing the lung cancer model by the method is hundred percent, and the atomization inhalation mode can better simulate a randomness of an internal occurrence site of the human lung cancer, with a high standardization degree.

Moreover, according to the method, all stages in the occurrence of the lung cancer can be constructed and stimulated by regulating the diameter of the atomized particle, using different genetically engineered mice, and controlling the inhalation of different viruses and the viruses with different concentrations, or the concentration of the chemical carcinogen. Meanwhile, the method can be applied to construction of different lung cancer models by the control means, such as the squamous carcinoma, the small cell lung cancer, etc.

The method has very important significance for studying the key nodes in the occurrence and development of the lung cancer and the effective target drug control.

### BRIEF DESCRIPTION OF THE DRAWINGS

FIG. 1 is a schematic diagram illustrating aerosol inhalation of virus.

FIG. 2 is a schematic diagram illustrating a generating process of a lung cancer after a genetically engineered mouse inhaled the virus. Kras^{LSL-G12D} mouse (No. #008179, Jackson Laboratory)

FIG. 3 illustrates occurrence and development of the lung cancer dynamically observed by small animal CT imaging.

FIG. 4 illustrates a lung cancer-hypermetabolism area observed by small animal PET/CT imaging.

FIG. 5 illustrates the occurrence and development of the lung cancer generally observed through a lung tissue.

FIG. 6 illustrates the occurrence and development of the lung cancer observed by tissue section upon HE staining.

### DETAILED DESCRIPTION

The present invention is further described hereinafter with reference to the drawings and specific embodiments of the description, but the embodiments do not limit the present invention in any form. Unless otherwise specified, the reagents, methods and devices used in the present invention are conventional reagents, methods and devices in the technical field.

Unless otherwise specified, the reagents and materials used in the following embodiments are commercially available.

### Embodiment 1 Construction of Lung Cancer Animal Model

1. Experimental Atomization Instrument

The atomization instrument is an atomization inhalation instrument Pari-3305-Junior boy SX purchased from Germany, with a mass median diameter (MMD) of 2.9 μm.

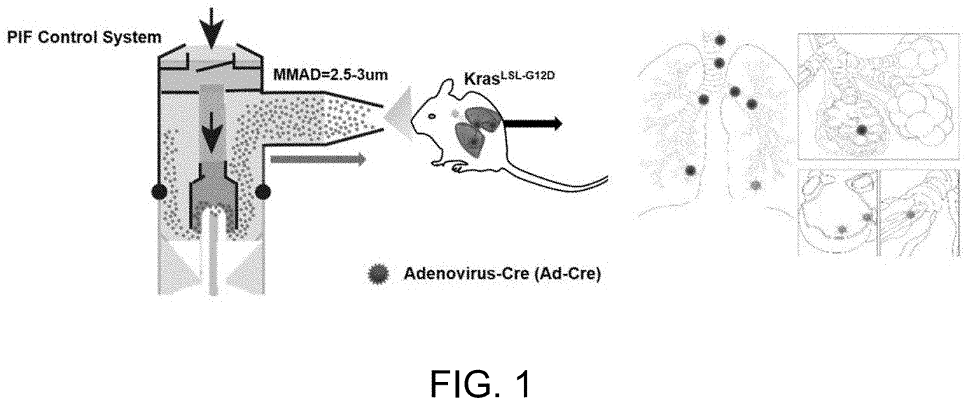

A schematic diagram illustrating aerosol inhalation of virus is shown in FIG. 1.

When in use, air was compressed to inhale and atomize a solution, a spray was generated by a compressor, and then transmitted to an atomizer through an air hose to spray, and a liquid aerosol was atomized and transmitted to a spray nozzle. An amount of the spray was increased by inhaling an additional air flow generated, so that the inhalation was performed quickly and effectively.

The atomizer was provided with a PIF control system to help slow absorption, so that atomized particles were uniformly distributed in a bronchiole and an alveolus.

2. Experimental Mice

Kras^{LSL-G12D} genetically engineered mice aged 8 weeks to 16 weeks (No. #008179, Jackson Laboratory) were used.

An animal experiment program was approved by the Animal Experiment Ethics Committee of Sun Yat-sen University and Southern Medical University. All the animal experiments conformed to the Guide for the Care and Use of Laboratory Animals issued by National Institutes of Health (NIH Publication, Eighth Edition, 2011).

3. Experimental Virus Drug

Adenovirus carrying Cre recombinase (Shanghai Genechem).

4. A method for constructing a lung cancer animal model included the following steps.

In S1, an adenovirus stock solution with a titer of 8E+10 PFU/mL was diluted with PBS into working concentrations according to experimental requirements, which were: virus titers=5×10⁵ PFU/mL, 5×10⁶ PFU/mL, 2.5×10⁷ PFU/mL, 5×10⁷ PFU/mL, 5×10⁸ PFU/mL and 7.8×10⁹ PFU/mL respectively. An amount of a liquid in an atomization cup of the atomization inhalation instrument ranged from 2 ml to 3 ml.

In S2, an anesthetized mouse was stably placed on a stage and put on a mask, and the atomization inhalation instrument was started to enable the mouse to inhale atomized particles of the virus carrying the Cre recombinase for 15 minutes to 20 minutes. An atomization air flow was kept stable during the period until the liquid in the atomization cup of the atomization inhalation instrument was completely inhaled.

Parameters of the atomization inhalation instrument in the step S2 might be set as follows:

pressure: 0.5 bar/50 kpa to 2.0 bar/200 kpa

working flow: 3.0 L/min to 6.0 L/min

atmospheric pressure: 500 hpa to 1060 hpa

atomization rate: 370 mg/min

size of atomized particle: mean median diameter (MMAD): 2.9 μm; and percentage of particulate of <5 μm: 76%.

The atomized particles could be uniformly dispersed on an alveolar epithelium and enter the alveolar epithelium, wherein the Cre recombinase played a role to activate an oncogene, thus generating a lung cancer.

In S3, the mouse was placed in an SPF environment for observation after waking up, and small animal CT imaging was performed 2, 4 and 5 months after the inhalation to dynamically observe occurrence and development of a tumor. Tissue sampling was performed 4 and 5 months after the inhalation to observe occurrence and development of a lung tumor in situ until the model was successfully constructed.

5. Experimental Results

Since an inhalation delivery system provided an extremely small aerosol liquid drop carrying the virus to the alveolus, the Cre recombinase was expressed in an infected lung cell, and a Kras gene in the Kras^{LSL-G12D} mouse was activated, which finally caused the tumor in a lung.

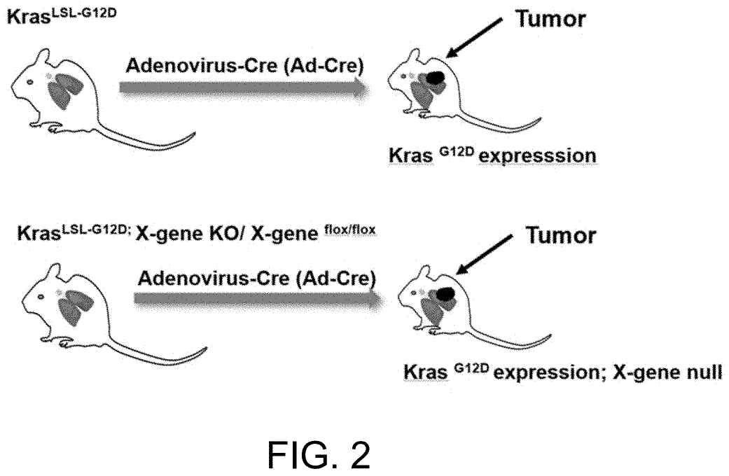

The occurrence and development of the tumor of the mouse inhaling the virus were dynamically observed by small animal CT and PET/CT. The mice were put to death at different time points for the tissue sampling, and the occurrence and development of the lung cancer were pathologically observed. The results were shown in FIG. 2 to FIG. 6.

FIG. 2 is a schematic diagram illustrating a generating process of a lung cancer after a genetically engineered mouse inhaled the virus. After the Kras^{LSL-G12D} mouse inhaled the adenovirus carrying the Cre recombinase, the Cre excised two loxP sites, causing failure of a termination codon, thus activating a downstream Kras oncogene. The activated Kras gene could cause the lung cancer of the mouse.

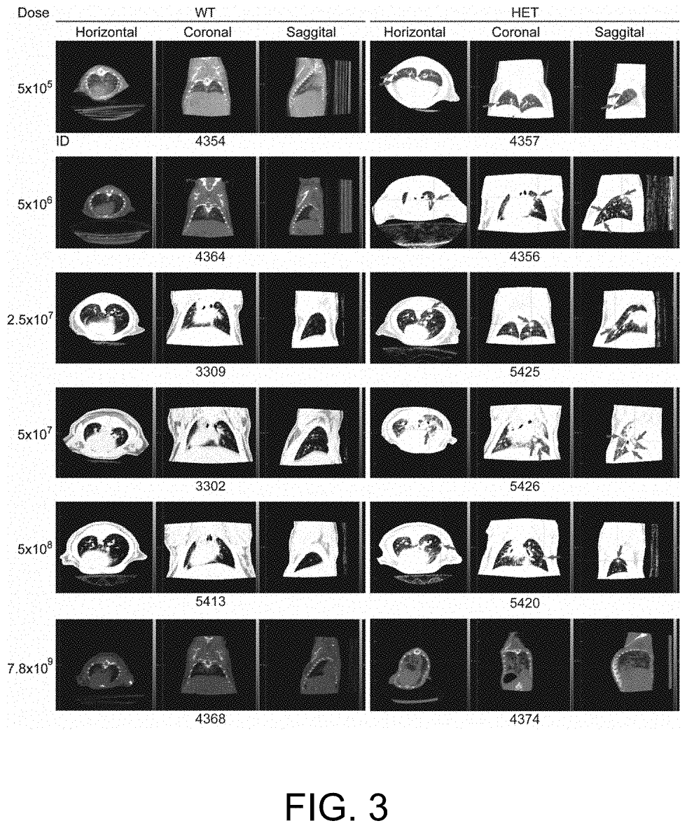

FIG. 3 illustrates occurrence and development of the lung cancer dynamically observed by mouse CT imaging. In lung scan images of a WT mouse (left, WT) and a Kras^{LSL-G12D} mouse (right, HET) inhaling and infected with the virus (titers were respectively 5×10⁵, 5×10⁶, 2.5×10⁷, 5×10⁷, 5×10⁸ and 7.8×10⁹), the tumor was a white high intensity area (marked with a circle and an arrow).

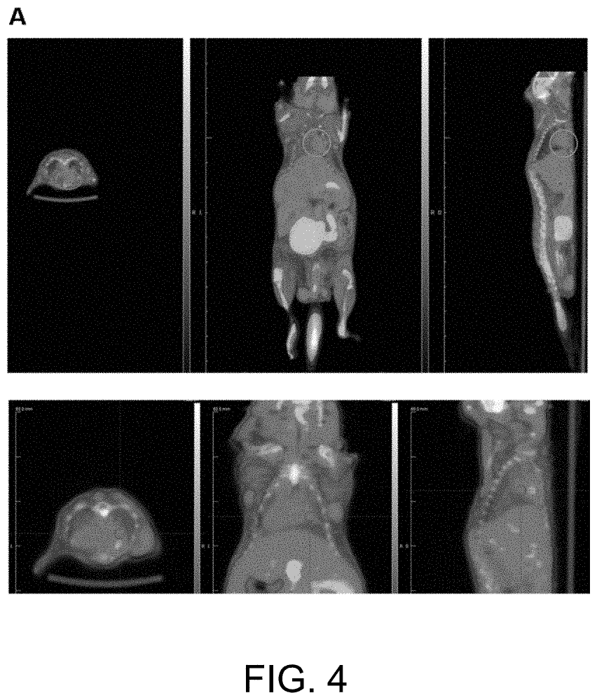

FIG. 4 illustrates a cancer-hypermetabolism area observed by mouse PET/CT imaging for monitoring a metabolic activity of the tumor. The Kras^{LSL-G12D} mouse was scanned by PET/CT 16-20 weeks after inhaling the virus (7.8×10⁹). Compared with a control group, an increase in uptake of glucose F18 was locally shown in a left lung of the mouse. Meanwhile, this area was suggested to be a tumor hypermetabolism area (see the circle mark) combined with CT results.

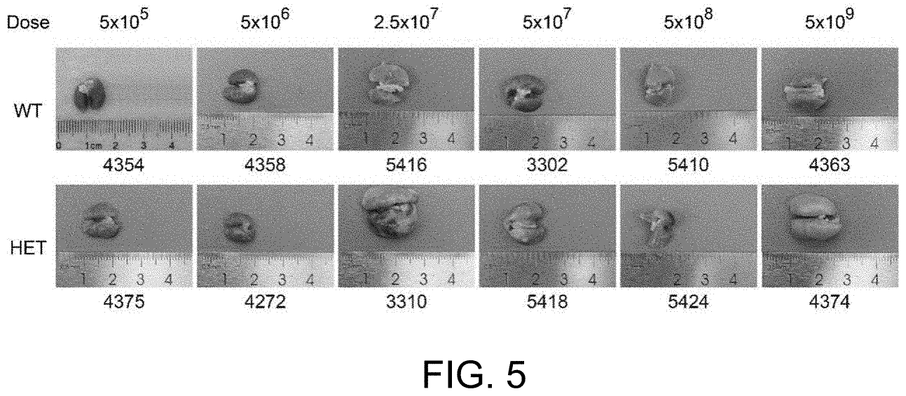

FIG. 5 illustrates the occurrence and development of the lung cancer generally observed by sampling a lung tissue, including occurrence of lung tumors of the WT mouse and the Kras^{LSL-G12D} mouse inhaling and infected with the virus (titers are respectively 5×10⁵, 5×10⁶, 2.5×10⁷, 5×10⁷, 5×10⁸ and 7.8×10⁹).

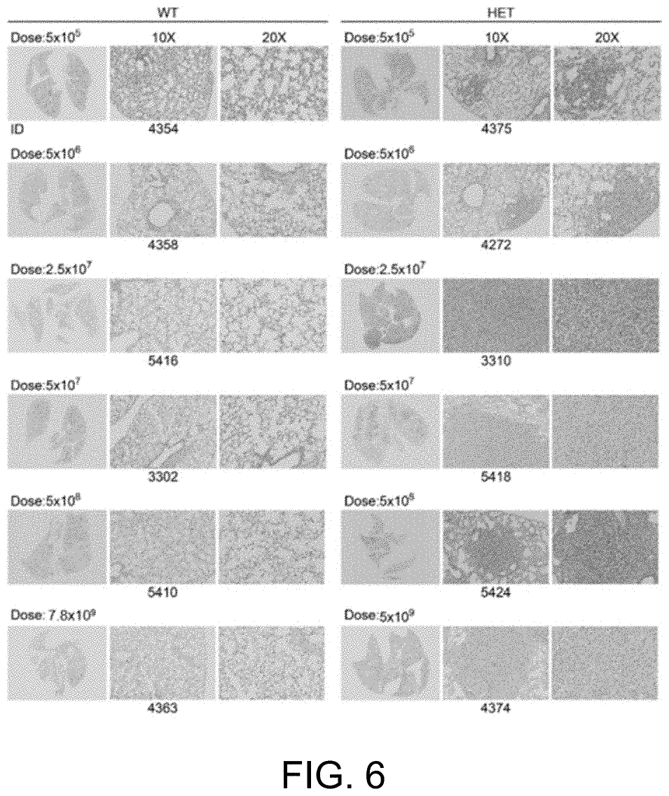

FIG. 6 illustrates the occurrence and development of the lung cancer observed by tissue section upon HE staining, including the occurrence of the lung tumors of the WT mouse (left, WT) and the Kras^{LSL-G12D} mouse (right, HET) inhaling and infected with the virus (titers are respectively 5×10⁵, 5×10⁶, 2.5×10⁷, 5×10⁷, 5×10⁸ and 7.8×10⁹). According to results of histopathological analysis, an early stage of the lung cancer was stimulated at a virus concentration of 5×10⁵-5×10⁶. A progressing stage of the lung cancer was stimulated at a virus concentration of 2.5×10⁷. An invasive carcinoma stage of the lung cancer was stimulated at a virus concentration of 7.8×10⁹.

The above embodiments are preferred embodiments of the present invention, but the embodiments of the present invention are not limited by the above embodiments. Any other changes, modifications, substitutions, combinations and simplifications made without deviating from the spiritual substance and principle of the present invention shall be equivalent substitute modes, and are all included in the protection scope of the present invention.

1. A method for constructing a lung cancer animal model, comprising an animal subjected to a nebulization after a pathogenic substance of a lung cancer is atomized into atomized particles, so that the animal inhales the atomized particles in an inhaling mode.

2. The construction method according to claim 1, wherein a size of the atomized particle is that: a mass median aerodynamic diameter (MMAD) is 2.9 μm; and a percentage of a particulate smaller than 5 μm is 76%.

3. The construction method according to claim 1, wherein the nebulization is for the animal to continuously inhale for 15 minutes to 20 minutes.

4. The construction method according to claim 1, wherein an atomization amount of the pathogenic substance of the lung cancer ranges from 2 ml to 8 ml.

5. The construction method according to claim 1, wherein after inhaling the atomized particles, the animal is placed in an SPF environment to obtain a required lung cancer animal model.

6. The construction method according to claim 1, wherein the animal is subjected to the nebulization after the pathogenic substance of the lung cancer is atomized into the atomized particles by using an atomization inhalation instrument, and operating parameters of the instrument are as follows:
pressure: 0.5 bar/50 kpa to 2.0 bar/200 kpa
atomization amount: 2 ml to 8 ml
working flow: 3.0 L/min to 6.0 L/min
atmospheric pressure: 500 hpa to 1060 hpa
atomization rate: 370 mg/min
size of particle: mass median aerodynamic diameter (MMAD): 2.9 μm; and percentage of particulate smaller than 5 μm: 76%.

7. The construction method according to claim 1, wherein in the case that a diameter of the atomized particle ranges from 5 μm to 10 μm, the obtained animal model simulates the lung cancer occurring on a main bronchus and a secondary bronchus, in the case that the diameter ranges from 3 μm to 5 μm, the obtained animal model simulates the lung cancer occurring on the secondary bronchus and branched bronchi, and in the case that the diameter is less than 3 μm, the obtained animal model simulates the lung cancer occurring on a terminal bronchiolar epithelium and an alveolar epithelium.

8. The construction method according to claim 7, wherein when the diameter of the atomized particle ranges from 2 μm to 3 μm, the constructed animal model simulates an adenocarcinoma in non-small cell lung cancers.

9. The construction method according to claim 8, wherein the pathogenic substance of the lung cancer is an adenovirus carrying a Cre recombinase capable of activating a Kras oncogene of a lung epithelial cell; in the case that a virus concentration is 5×10⁵-5×10⁶, the obtained animal model simulates an early stage of the lung cancer; in the case that the virus concentration is 2.5×10⁷, the obtained animal model simulates a progressing stage of the lung cancer; and in the case that the virus concentration is 7.8×10⁹, the obtained animal model simulates an invasive carcinoma stage of the lung cancer.

10. The construction method according to claim 7, wherein in the case that the pathogenic substance of the lung cancer is an adenovirus carrying a Cre recombinase, and the diameter of the atomized particle ranges from 5 μm to 10 μm, the atomized particles are inhaled by Kras^{LSL-G12D};LKB1^{fl/fl} genetically engineered mice, which are hybrids of Kras^{LSL-G12D} mice and LKB1^{fl/fl} mice, to construct mouse models of a squamous carcinoma in non-small cell lung cancers.

11. The construction method according to claim 2, characterized in that, the nebulization is for the animal to continuously inhale for 15 minutes to 20 minutes.
